(19) United States
(12) Patent Application Publication
(10) Pub. No.: US 2022/0249716 A1
(43) Pub. Date: Aug. 11, 2022

(54) LAUNDRY PEDESTAL WITH ULTRAVIOLET STERILIZATION DRAWER

(71) Applicant: Haier US Appliance Solutions, Inc., Wilmington, DE (US)

(72) Inventors: Srikanth Raavi Venkata, Louisville, KY (US); Abdel Hamad, Louisville, KY (US); Kyle James Tear, Louisville, KY (US)

(21) Appl. No.: 17/173,373

(22) Filed: Feb. 11, 2021

(51) Int. Cl. A61L 2/10; A61L 2/24; D06F 29/00; D06F 58/20

(52) U.S. Cl. CPC A61L 2/10; A61L 2/24; D06F 29/00; A61L 2202/26; A61L 2202/122; A61L 2202/11; A61L 2202/14; D06F 58/20

(57) ABSTRACT

A laundry appliance system includes a cabinet that is supported by a pedestal defining a pedestal chamber. A sterilization module is mounted within the pedestal chamber for sterilizing unsterilized items placed into the pedestal chamber. The sterilization module includes a sterilization housing defining a sterilization chamber and a sterilization light source, such as a UV-C ultraviolet light, that is positioned therein for illuminating the sterilization chamber. One or more walls may be positioned within the sterilization chamber to prevent direct line of sight of the sterilization light source from a housing inlet or outlet and an air handler is positioned in the sterilization housing for urging a flow of air through the sterilization chamber so that the flow of air is sterilized by the sterilization light source.

### FIELD OF THE INVENTION

The present subject matter relates generally to laundry appliances, and more particularly to pedestals and object sterilization systems for use with laundry appliances.

### BACKGROUND OF THE INVENTION

Laundry appliances, such as washing machine appliances and dryer appliances, typically include an external apron or cabinet that acts as a frame for supporting various components of the appliance and a housing that provides a clean look and prevents a user from contacting internal components. In order to provide access to the controls and chambers of the laundry appliance, certain laundry systems are also compatible with a riser pedestal on which the appliance cabinet sits during operation. These pedestals provide extra storage below the appliance as well as provide access to the appliance without bending over. For example, conventional pedestals include a sliding drawer that may be used to store detergent or other laundry-related items. However, these sliding drawers are often underutilized or not used at all.

In addition, common households contain many items which could benefit from periodic sterilization for a safer and healthier home environment. For example, toys, jackets, throws, blankets, rugs, and other infrequently washed items could benefit from periodic cleaning or sterilization. In addition, food such as fruits or vegetables may benefit from selective sterilization. Moreover, delivery boxes, kitchen cutlery, and other common household items may frequently be contaminated with fungus, bacteria, mold, or other undesirable contaminants such that the implementation of a sterilization process might be desirable.

Accordingly, a laundry appliance system with improved utilization of laundry pedestals would be useful. More specifically, a pedestal that may be used to facilitate sterilization or sanitization of common household items that are infrequently washed would be particularly beneficial.

### BRIEF DESCRIPTION OF THE INVENTION

Aspects and advantages of the invention will be set forth in part in the following description, or may be obvious from the description, or may be learned through practice of the invention.

In one exemplary embodiment, a laundry appliance is provided including a cabinet, a basket rotatably mounted within the cabinet and defining a chamber configured for receiving a load of clothes, a pedestal for supporting the cabinet, the pedestal defining a pedestal chamber for receiving unsterilized items, and a sterilization module positioned within the pedestal chamber for selectively sterilizing the unsterilized items. The sterilization module includes a sterilization housing defining a sterilization chamber, a sterilization light source positioned within the sterilization chamber for illuminating the sterilization chamber, and an air handler for urging a flow of air through the sterilization chamber so that the flow of air is sterilized by the sterilization light source.

In another exemplary embodiment, a sterilization module for a pedestal of a laundry appliance is provided. The pedestal defines a pedestal chamber for receiving unsterilized items and the sterilization module includes a sterilization housing defining a sterilization chamber, a sterilization light source positioned within the sterilization chamber for illuminating the sterilization chamber, and an air handler for urging a flow of air through the sterilization chamber so that the flow of air is sterilized by the sterilization light source.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a washing machine appliance and pedestal assembly according to an exemplary embodiment of the present subject matter.

FIG. 2 provides a perspective view of the exemplary pedestal of FIG. 1 with a drawer of the exemplary pedestal shown in an open position.

FIG. 3 provides a perspective view of a pedestal chamber of the pedestal containing items to be sterilized by a plurality of sterilization modules according to an exemplary embodiment of the present subject matter.

FIG. 4 provides a perspective view of a sterilization module according to an exemplary embodiment of the present subject matter.

FIG. 5 provides a perspective, cross-sectional view of the exemplary sterilization module of FIG. 4 according to an exemplary embodiment of the present subject matter.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. For example, the approximating language may refer to being within a 10 percent margin.

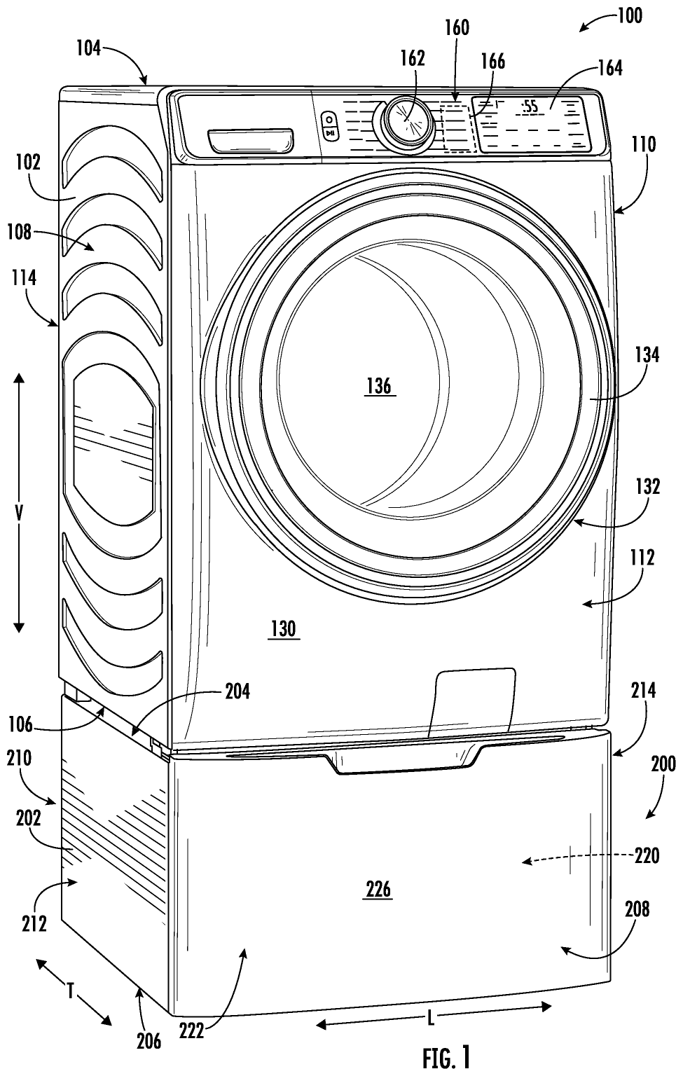

Referring now to the figures, an exemplary laundry appliance that may be used to implement aspects of the present subject matter will be described. Specifically, FIG. 1 is a perspective view of an exemplary horizontal axis washing machine appliance 100. As illustrated, washing machine appliance 100 generally defines a vertical direction V, a lateral direction L, and a transverse direction T, each of which is mutually perpendicular, such that an orthogonal coordinate system is generally defined. Washing machine appliance 100 includes a cabinet 102 that extends between a top 104 and a bottom 106 along the vertical direction V, between a left side 108 and a right side 110 along the lateral direction, and between a front 112 and a rear 114 along the transverse direction T.

Washing machine appliance 100 may include a wash basket that is rotatably mounted within cabinet 102 such that it is rotatable about an axis of rotation. A motor, e.g., such as a pancake motor, may be in mechanical communication with the wash basket to selectively rotate the wash basket (e.g., during an agitation or a rinse cycle of washing machine appliance 100). The wash basket is received within a wash tub and defines a wash chamber that is configured for receipt of articles for washing. The wash tub holds wash and rinse fluids for agitation in the wash basket within the wash tub. As used herein, “wash fluid” may refer to water, detergent, fabric softener, bleach, or any other suitable wash additive or combination thereof. Indeed, for simplicity of discussion, these terms may all be used interchangeably herein without limiting the present subject matter to any particular “wash fluid.”

The wash basket may define one or more agitator features that extend into the wash chamber to assist in agitation and cleaning articles disposed within the wash chamber during operation of washing machine appliance 100. For example, a plurality of ribs may extend from the wash basket into the wash chamber. In this manner, for example, the ribs may lift articles disposed in the wash basket during rotation of the wash basket.

The wash basket may also define a plurality of perforations in order to facilitate fluid communication between an interior of the wash basket and the wash tub. A sump is defined by the wash tub at a bottom of the wash tub along the vertical direction V. Thus, the sump is configured for receipt of and generally collects wash fluid during operation of washing machine appliance 100. For example, during operation of washing machine appliance 100, wash fluid may be urged by gravity from the wash basket to the sump through the plurality of perforations. A drain pump assembly is located beneath the wash tub and is in fluid communication with the sump for periodically discharging soiled wash fluid from washing machine appliance 100, e.g., via a drain hose to an external drain.

Referring still to FIG. 1, cabinet 102 also includes a front panel 130 which defines an opening 132 that permits user access to the wash basket or the wash chamber. More specifically, washing machine appliance 100 includes a door 134 that is positioned over opening 132 and is rotatably mounted to front panel 130. In this manner, door 134 permits selective access to opening 132 by being movable between an open position (not shown) facilitating access to the wash tub and a closed position (FIG. 1) prohibiting access to the wash tub. A window 136 in door 134 permits viewing of the wash basket when door 134 is in the closed position, e.g., during operation of washing machine appliance 100. Door 134 also includes a handle (not shown) that, e.g., a user may pull when opening and closing door 134. Further, although door 134 is illustrated as mounted to front panel 130, it should be appreciated that door 134 may be mounted to another side of cabinet 102 or any other suitable support according to alternative embodiments.

A control panel 160 including a plurality of input selectors 162 is coupled to front panel 130. Control panel 160 and input selectors 162 collectively form a user interface input for operator selection of machine cycles and features. For example, in one embodiment, a display 164 indicates selected features, a countdown timer, and/or other items of interest to machine users. Operation of washing machine appliance 100 is controlled by a controller or processing device 166 (FIG. 1) that is operatively coupled to control panel 160 for user manipulation to select washing machine cycles and features. In response to user manipulation of control panel 160, controller 166 operates the various components of washing machine appliance 100 to execute selected machine cycles and features.

Controller 166 may include a memory and microprocessor, such as a general or special purpose microprocessor operable to execute programming instructions or micro-control code associated with a cleaning cycle. The memory may represent random access memory such as DRAM, or read only memory such as ROM or FLASH. In one embodiment, the processor executes programming instructions stored in memory. The memory may be a separate component from the processor or may be included onboard within the processor. Alternatively, controller 166 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software. Control panel 160 and other components of washing machine appliance 100 may be in communication with controller 166 via one or more signal lines or shared communication busses.

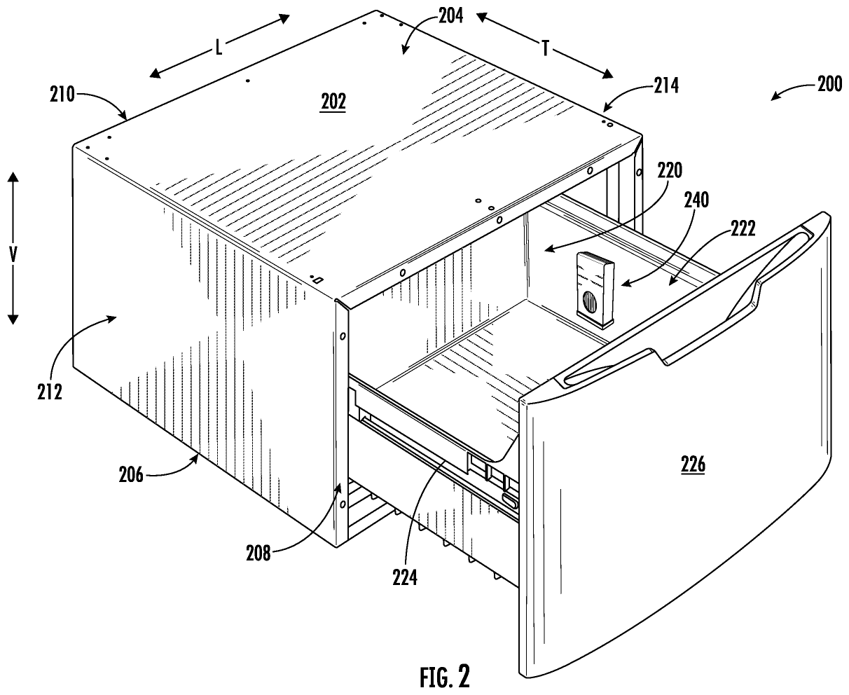

Referring now to FIGS. 1 and 2, washing machine appliance 100 may include a pedestal 200 that is configured for elevating or raising the height of washing machine appliance 100 to a height more convenient for a user or to provide storage space underneath the appliance. Although pedestal 200 will be described herein as being used with washing machine appliance 100, it should be appreciated that washing machine appliance 100 is used in order to facilitate discussion regarding the use and operation of pedestal 200. However, it should be appreciated that pedestal 200 and the sterilization assemblies described herein may be used with any other suitable laundry appliance, such as a dryer appliance, a combination washer/dryer appliance, etc. It should be further appreciated that the present subject matter is not limited to the style or configuration of the appliances described herein.

Pedestal 200 will now be described in more detail according to an exemplary embodiment of the present subject matter. In general, pedestal 200 is used for raising washing machine appliance 100 (or a dryer appliance). As illustrated, pedestal 200 includes a box or housing 202 having a substantially rectangular cross-section that extends between a first side or top side 204 and a second side or bottom side 206 along the vertical direction V. Although the terms “top” and “bottom” are used to refer to sides 204, 206, it should be appreciated that such terminology is only intended to facilitate discussion of the present subject matter, and is not intended to restrict the directional orientation of pedestal 200. In addition, pedestal 200 generally extends between a front side 208 and a rear side 210 along the transverse direction T, and between a left side 212 and right side 214 along the lateral direction L.

According to exemplary embodiments, pedestal 200 may be formed from any material which is sufficiently rigid to support washing machine appliance 100 (or a dryer appliance). For example, pedestal 200 may be formed by injection molding, e.g., using a suitable plastic material, such as injection molding grade high impact polystyrene (HIPS) or acrylonitrile butadiene styrene (ABS). Alternatively, according to the exemplary embodiment, these components may be compression molded, e.g., using sheet molding compound (SMC) thermoset plastic or other thermoplastics. According still other embodiments, pedestal 200 may be formed from metal or any other suitable rigid material, such as sheet metal.

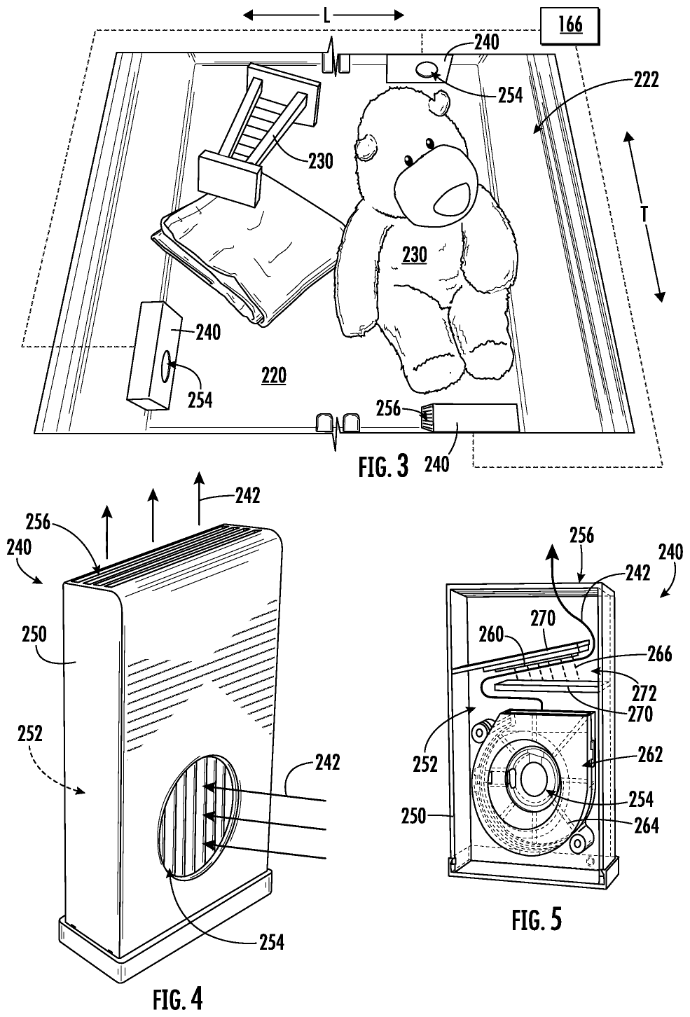

As best shown in FIGS. 2 and 3, pedestal 200 generally includes or defines a pedestal chamber 220, e.g., within housing 202. Notably, as explained above, conventional riser pedestals are typically configured for storage of detergent or other laundry supplies. However, the storage area within these pedestals is often underutilized. As a result, aspects of the present subject matter are directed to a system and method for better utilizing the space within a laundry pedestal, e.g., by providing for the sterilization of household items or other objects.

According to the illustrated embodiment, pedestal 200 further includes a drawer 222 that is slidably mounted within housing 202 using one or more slide assemblies 224. A drawer front 226 is mounted at the front end of drawer 222 and may include a handle to permit a user to grab drawer 222 and slide it into and out of pedestal chamber 220. Although pedestal 200 is illustrated herein as including a drawer 222, it should be appreciated that according to alternative embodiments, pedestal 200 may include a door or other suitable means for providing selective access to pedestal chamber 220.

In general, pedestal chamber 220 may be generally configured for receiving unsterilized items (e.g., as identified generally by reference numeral 230 in FIG. 3). In general, unsterilized items 230 may be any object or item that a user of washing machine appliance 100 wishes to sanitize, clean, disinfect, or otherwise sterilize. For example, unsterilized items 230 may include children's toys, jackets, towels, blankets, throws, delivered boxes, mail, food items, utensils, or any other common household items that are preferably sterilized. To perform a sterilization cycle, a user may simply place unsterilized items 230 within drawer 222 and close drawer 222 by sliding it into pedestal chamber 220. By contrast, when pedestal 200 utilizes a door enclosure, a user may simply place unsterilized items 230 within pedestal chamber 220 before closing the door.

A system and method for sterilizing unsterilized items 230 within pedestal chamber 220 will now be described according to exemplary embodiments of the present subject matter. Specifically, as shown for example in FIG. 3, pedestal 200 may include one or more sterilization modules 240 that are positioned within pedestal chamber 220 and are configured for sterilizing items placed therein. Specifically, according to the illustrated embodiment, pedestal 200 includes three sterilization modules 240 positioned on various walls of drawer 222. However, it should be appreciated that according to alternative embodiments, any suitable number, type, position, and configuration of sterilization modules 240 may be used while remaining within the scope of the present subject matter. An exemplary sterilization module 240 and its method of operation will be described below according to an exemplary embodiment. However, it should be appreciated that variations and modifications may be made to sterilization module 240 while remaining within scope the present subject matter.

Sterilization module 240 is generally configured for sterilizing the air (identified generally by reference numeral 242) within pedestal chamber 220, thereby reducing the existence of bacteria, mold, fungus, pathogens, or other contaminants within the air 242. Notably, by sanitizing or sterilizing air 242 within pedestal chamber 220 in this manner, unsterilized items 230 that are positioned within pedestal chamber 220 are also sterilized by constant exposure to sterilized air 242. Thus, sterilization modules 240 are generally configured for drawing in contaminated or unsterilized air before sanitizing that air and discharging it back into pedestal chamber 220. As a result, a sanitized or sterilized environment is developed within pedestal chamber 220 while sterilization modules 240 are running.

As illustrated, sterilization module 240 generally includes a sterilization housing 250 that defines a sterilization chamber 252 therein. Sterilization housing 250 may further define an inlet 254 through which a flow of air 242 is drawn into sterilization chamber 252 from pedestal chamber 220. As will be described in more detail below, the sterilization process is performed within sterilization chamber 252 prior to discharging the flow of sterilized air 242 back into pedestal chamber 220 through an outlet 256 defined in sterilization housing 250. According to the illustrated embodiment, inlet 254 is a circular inlet defined on a side of sterilization housing 250 and outlet 256 is a rectangular outlet defined on an end of sterilization housing 250. However, it should be appreciated that according to alternative embodiments, inlet 254 and outlet 256 may have any other suitable size, position, and geometry for facilitating the desired flow of air 242 through sterilization housing 250.

Sterilization module 240 may further include a sterilization light source 260 that is positioned within sterilization chamber 252 for selectively illuminating sterilization chamber 252 and sterilizing the flow of air 242 passing therethrough. In addition, sterilization module may include an air handler 262 for selectively urging the flow of air 242 through sterilization chamber 252 so that the flow of air may be constantly sterilized by sterilization light source 260. According to the illustrated embodiment, air handler 262 is a centrifugal fan 264 that is mounted over inlet 254 and is positioned within sterilization housing 250. However, it should be appreciated that according to alternative embodiments, any suitable mechanism for urging a flow of air 242 through sterilization housing 250 may be used. For example, axial fans, tangential fans, or any other suitable air handling device positioned and configured in any other suitable manner may be used.

In general, sterilization light source 260 may generally be configured for illuminating sterilization chamber 252 with sterilizing light (e.g., as identified generally in FIG. 5 by reference numeral 266). In this regard, sterilization light source 260 may include any suitable number, type, and configuration of light sources for generating sterilizing light 266. According to the exemplary illustrated embodiment, sterilization light source includes one or more elongated light sources containing one or more light emitting diodes (LEDs). For example, according to exemplary embodiments, sterilization light source 260 includes ultraviolet (UV) lights. Indeed, according to the illustrated exemplary embodiment, sterilization light source 260 includes C-band ultraviolet lights, i.e., UV-C light. According to exemplary embodiments, sterilization light source 260 may generate ultraviolet light having a wavelength of between 50 and 500 nanometers, between 100 and 400 nanometers, between 200 and 300 nanometers, or between about 265 and 280 nanometers. In addition, it should be appreciated that sterilization light source 260 may include different types of lights producing a broad spectrum of sterilizing light 266 as needed for a given application.

According to exemplary embodiments, a controller (e.g., such as controller 166 of washing machine appliance 100 or another suitable dedicated controller) may regulate the operation of sterilization module 240 to initiate and perform a sterilization cycle. More specifically, controller 166 may periodically or selectively energize centrifugal fan 264 and illuminate sterilization light source 260 in order to circulate and sterilize the flow of air 242 and create a sterilized or sanitized environment within pedestal chamber 220. For example, controller 166 may be configured to determine when a sanitization or sterilization cycle is needed and perform the sterilization cycle. In this regard, controller 166 may determine that unsterilized items 230 have been placed in pedestal chamber 220 and may initiate a sterilization cycle to sterilize those items. Controller 166 may use any suitable means for detecting the presence of unsterilized items 230 and/or the need for a sterilization cycle, e.g., such as an object detection system. According to alternative exemplary embodiments, controller 166 may be configured for performing periodic sterilization cycles regardless of whether unsterilized items 230 are present. In addition, controller 166 may be configured for performing a sterilization cycle on demand, e.g., as selected by a user via control panel 160 of washing machine appliance 100. The duration, light intensity, and other sterilization cycle parameters may be adjusted as needed depending on the application to improve the sterilization process.

Notably, human exposure to sterilization light 266 may typically be undesirable. For example, particularly when sterilization light 266 includes electromagnetic energy having harmful wavelengths, such as the UVC light, it is desirable to prevent direct exposure of a user to such light. As a result, sterilization module 240 may include features for enclosing sterilization light source 260 and isolating sterilizing light 266 to prevent user exposure.

In this regard, for example, sterilization module 240 may include one or more internal walls 270 that are positioned within sterilization housing 250 between inlet 254 and outlet 256. According to exemplary embodiments, internal walls 270 may generally define an internal passageway 272 within sterilization chamber 252 through which the entire flow of air 242 is passed. In this regard, internal walls 270 are illustrated in FIG. 5 as being substantially parallel walls that define a narrow internal passageway 272 that directs all of the flow of air 242 in close proximity to sterilization light source 260. However, it should be appreciated that any suitable number, position, and configuration of internal walls 270 may be used according to alternative embodiments.

Notably, internal walls 270 may generally prevent direct line of sight into the internal passageway 272. Specifically, internal walls 270 may be sized and positioned to prevent a user from viewing internal passageway 272 or sterilization light source 260 directly from inlet 254 or outlet 256. For example, as illustrated, sterilization light source 260 is mounted within the internal passageway 272 on at least one of internal walls 270. In addition, internal walls 270 may extend across substantially the entire width of sterilization housing 250 to form a zigzag pattern and may be positioned directly between inlet 254 and outlet 256 such that a user cannot view sterilization light source 260 without disassembling sterilization housing 250. In this manner, sterilization module 240 may be safely operated to implement a sterilization cycle without exposing the user to harmful sterilizing light 266. In addition, because of the inherently safe operation of sterilization module 240, door locks or restrictive operating cycles are not necessary to ensure user safety.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A laundry appliance comprising:
a cabinet;
a basket rotatably mounted within the cabinet and defining a chamber configured for receiving a load of clothes;
a pedestal for supporting the cabinet, the pedestal defining a pedestal chamber for receiving unsterilized items; and
a sterilization module positioned within the pedestal chamber for selectively sterilizing the unsterilized items, the sterilization module comprising:
a sterilization housing defining a sterilization chamber;
a sterilization light source positioned within the sterilization chamber for illuminating the sterilization chamber; and
an air handler for urging a flow of air through the sterilization chamber so that the flow of air is sterilized by the sterilization light source.

2. The laundry appliance of claim 1, wherein the sterilization housing comprises:
an inlet;
an outlet; and
one or more internal walls positioned between the inlet and the outlet, the one or more internal walls defining an internal passageway within sterilization chamber for receiving the flow of air.

3. The laundry appliance of claim 2, wherein the one or more internal walls prevent direct line of sight into the internal passageway from the inlet or the outlet of the sterilization housing.

4. The laundry appliance of claim 2, wherein the sterilization light source is mounted on within the internal passageway on at least one of the one or more internal walls.

5. The laundry appliance of claim 1, wherein the sterilization light source comprises:
a light emitting diode.

6. The laundry appliance of claim 1, wherein the sterilization light source comprises:
an ultraviolet light.

7. The laundry appliance of claim 6, wherein the ultraviolet light is UV-C light having a wavelength between 265 and 280 nanometers.

8. The laundry appliance of claim 1, wherein the laundry appliance comprises two or more sterilization modules positioned within the pedestal chamber.

9. The laundry appliance of claim 1, further comprising:
a drawer or door mounted to the pedestal for providing selective access to the pedestal chamber.

10. The laundry appliance of claim 9, wherein the sterilization module is mounted on a side or a bottom of the drawer or to an inner surface of the door.

11. The laundry appliance of claim 1, wherein the air handler is a centrifugal fan positioned within the sterilization housing.

12. The laundry appliance of claim 1, wherein a controller is operably coupled to the sterilization module, the controller being configured to:
determine that the unsterilized items are present within the pedestal chamber; and
operate the sterilization module by activating the air handler and the sterilization light source to circulate and sterilize the flow of air from within the pedestal chamber.

13. The laundry appliance of claim 1, wherein the laundry appliance is a washing machine appliance or a dryer appliance.

14. A sterilization module for a pedestal of a laundry appliance, the pedestal defining a pedestal chamber for receiving unsterilized items, the sterilization module comprising:
a sterilization housing defining a sterilization chamber;
a sterilization light source positioned within the sterilization chamber for illuminating the sterilization chamber; and
an air handler for urging a flow of air through the sterilization chamber so that the flow of air is sterilized by the sterilization light source.

15. The sterilization module of claim 14, wherein the sterilization housing comprises:
an inlet;
an outlet; and
one or more internal walls positioned between the inlet and the outlet, the one or more internal walls defining an internal passageway within sterilization chamber for receiving the flow of air.

16. The sterilization module of claim 15, wherein the one or more internal walls prevent direct line of sight into the internal passageway from the inlet or the outlet of the sterilization housing.

17. The sterilization module of claim 15, wherein the sterilization light source is mounted on within the internal passageway on at least one of the one or more internal walls.

18. The sterilization module of claim 14, wherein the sterilization light source comprises:
an ultraviolet light.

19. The sterilization module of claim 18, wherein the ultraviolet light is UV-C light having a wavelength between 265 and 280 nanometers.

20. The sterilization module of claim 14, wherein a controller is operably coupled to the sterilization module, the controller being configured to:
determine that the unsterilized items are present within the pedestal chamber; and
operate the sterilization module by activating the air handler and the sterilization light source to circulate and sterilize the flow of air from within the pedestal chamber.
